(19) United States
(12) Patent Application Publication
(10) Pub. No.: US 2006/0228709 A1
(43) Pub. Date: Oct. 12, 2006

(54) Novel fibulin-like polypeptides

(75) Inventors: Jadwiga Bienkowska, Cambridge, MA (US); Gregg Mcallister, Charlestown, MA (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Applied Research Systems ARS Holding N.V., Curacao (NL)

(21) Appl. No.: 10/540,846

(22) PCT Filed: Dec. 10, 2003
(86) PCT No.: PCT/EP03/50974

§ 371 Date: Apr. 21, 2006

Related U.S. Application Data
(60) Provisional application No. 60/436,786, filed on Dec. 27, 2002.

(51) Int. Cl. C12Q 1/68; G01N 33/53; A01K 67/033; C07H 21/04; C12P 21/06; C07K 14/705; C07K 16/28; A61K 38/17

(52) U.S. Cl. 435/6; 530/350; 530/388.22; 435/69.1; 435/7.1; 435/320.1; 435/325; 536/23.5; 514/12; 800/8

(57) ABSTRACT

The present invention discloses open reading frames (ORFs) in human genome encoding for novel fibulin-like polypeptides, and reagents related thereto including variants, mutants and fragments of said polypeptides, as well as ligands and antagonists directed against them. The invention provides methods for identifying and making these molecules, for preparing pharmaceutical compositions containing them, and for using them in the diagnosis, prevention and treatment of diseases.

### FIELD OF THE INVENTION

The present invention relates to nucleic acid sequences identified in human genome as encoding for novel polypeptides, more specifically for fibulin-like polypeptides. All publications, patents and patent applications cited herein are incorporated in full by reference.

### BACKGROUND OF THE INVENTION

Many novel polypeptides have been already identified by applying strict homology criteria to known polypeptides of the same family. However, since the actual content in polypeptide-encoding sequences in the human genome for fibulin-like polypeptides (and for any other protein family) is still unknown, the possibility still exists to identify DNA sequence encoding polypeptide having fibulin-like polypeptide activities by applying alternative and less strict homology/structural criteria to the totality of Open Reading Frames (ORFs, that is, genomic sequences containing consecutive triplets of nucleotides coding for amino acids, not interrupted by a termination codon and potentially translatable in a polypeptide) present in the human genome.

The ability for cells to make and secrete extracellular proteins is central to many biological processes. Enzymes, growth factors, extracellular matrix proteins and signalling molecules are all secreted by cells. This is through fusion of a secretory vesicle with the plasma membrane. In most cases, but not all, proteins are directed to the endoplasmic reticulum and into secretory vesicles by a signal peptide. Signal peptides are cis-acting sequences that affect the transport of polypeptide chains from the cytoplasm to a membrane bound compartment such as a secretory vesicle. Polypeptides that are targeted to the secretory vesicles are either secreted into the extracellular matrix or are retained in the plasma membrane. The polypeptides that are retained in the plasma membrane will have one or more transmembrane domains. Examples of secreted proteins that play a central role in the functioning of a cell are cytokines, hormones, extracellular matrix proteins (adhesion molecules), proteases, and growth and differentiation factors.

The fibulins are a newly emerging family of secreted glycoproteins. The functions of the fibulins are not fully understood, but fibulins have been found in association with extracellular matrix structures such as connective tissue fibers, basement membranes and blood clots (see, for example, Miosge et al, 1996, Histochem J. 28:109-16; Pan et al., 1993, J. Cell Biol. 123:1269-77; Reinhardt et al., 1996, J Biol. Chem. 271:19489-96). These associations are attributed to the ability of fibulins to interact with other extracellular matrix proteins such as fibronectin, laminins, nidogen, perlecan, fibrillin and fibrinogen.

The roles that fibulins have in the formation and/or stabilization of extracellular matrix structures as well as their effects on cellular behavior are currently under investigation. However, it is clear that the identification of novel fibulin-like proteins is of significant importance in increasing understanding of the underlying pathways that lead to certain disease states in which these proteins are implicated, and in developing more effective gene or drug therapies to treat these disorders.

### SUMMARY OF THE INVENTION

The invention is based upon the identification of an Open Reading Frame (ORF) in the human genome encoding a novel fibulin-like polypeptide. This polypeptide will be referred to herein as the SCS0007 polypeptide.

Accordingly, the invention provides isolated SCS0007 polypeptides having the amino acid sequence given by SEQ ID NO: 2 and their mature forms (including SEQ ID NO:4), variants, and fragments, as polypeptides having the activity of fibulin-like polypeptides. The invention includes also the nucleic acids encoding them, vectors containing such nucleic acids, and cell containing these vectors or nucleic acids, as well as other related reagents such as fusion proteins, ligands, and antagonists.

The invention provides methods for identifying and making these molecules, for preparing pharmaceutical compositions containing them, and for using them in the diagnosis, prevention and treatment of diseases.

### BRIEF DESCRIPTION OF THE FIGURES

FIG. 1: alignment of the SCS0007 ORF with known related polypeptide sequences

FIG. 2: alignment of the SCS0007 ORF with other known related polypeptide sequences

### DETAILED DESCRIPTION OF THE INVENTION

According to a first aspect of the present invention there is provided an isolated polypeptide having fibulin-like activity selected from the group consisting of:
a) the amino acid sequence as recited in SEQ ID NO: 2;
b) the mature form of the polypeptide whose sequence is recited in SEQ ID NO: 2 (SEQ ID NO:4);
c) a variant of the amino acid sequence recited in SEQ ID NO: 2, wherein any amino acid specified in the chosen sequence is non-conservatively substituted, provided that no more than 15% of the amino acid residues in the sequence are so changed;
d) an active fragment, precursor, salt, or derivative of the amino acid sequences given in a) to c).

The novel polypeptide described herein was identified on the basis of a consensus sequence for human fibulin-like polypeptides in which the number and the positioning of selected amino acids are defined for a protein sequence having a length comparable to known fibulin-like polypeptides.

The totality of amino acid sequences obtained by translating the known ORFs in the human genome were challenged using this consensus sequence, and the positive hits were further screened for the presence of predicted specific structural and functional “signatures” that are distinctive of a polypeptide of this nature, and finally selected by comparing sequence features with known fibulin-like polypeptides. Therefore, the novel polypeptides of the invention can be predicted to have fibulin-like activities.

The terms “active” and “activity” refer to the fibulin-like properties predicted for the fibulin-like polypeptide whose amino acid sequence is presented in SEQ ID NO: 2 in the present application. These properties include the ability to bind to calcium ions and to proteins such as fibronectin.

In a second aspect, the invention provides a purified nucleic acid molecule which encodes a polypeptide of the first aspect of the invention. Preferably, the purified nucleic acid molecule has the nucleic acid sequence as recited in SEQ ID NO:1 (encoding the fibulin-like polypeptide whose amino acid sequence is recited in SEQ ID NO:2), SEQ ID NO:1a (the SCS0007 coding sequence) or SEQ ID NO:3 (encoding the mature form of this polypeptide, whose amino acid sequence is recited in SEQ ID NO:2).

In a third aspect, the invention provides a purified nucleic acid molecule which hydridizes under high stringency conditions with a nucleic acid molecule of the second aspect of the invention.

In a fourth aspect, the invention provides a vector, such as an expression vector, that contains a nucleic acid molecule of the second or third aspect of the invention.

In a fifth aspect, the invention provides a host cell transformed with a vector of the fourth aspect of the invention.

In a sixth aspect, the invention provides a ligand which binds specifically to, and which preferably inhibits the fibulin-like activity of a polypeptide of the first aspect of the invention. Ligands to a polypeptide according to the invention may come in various forms, including natural or modified substrates, enzymes, receptors, small organic molecules such as small natural or synthetic organic molecules of up to 2000Da, preferably 800Da or less, peptidomimetics, inorganic molecules, peptides, polypeptides, antibodies, structural or functional mimetics of the aforementioned.

In a seventh aspect, the invention provides a compound that is effective to alter the expression of a natural gene which encodes a polypeptide of the first aspect of the invention or to regulate the activity of a polypeptide of the first aspect of the invention.

A compound of the seventh aspect of the invention may either increase (agonise) or decrease (antagonise) the level of expression of the gene or the activity of the polypeptide. Importantly, the identification of the function of the fibulin-like polypeptide of the invention allows for the design of screening methods capable of identifying compounds that are effective in the treatment and/or diagnosis of disease.

In an eighth aspect, the invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in therapy or diagnosis. These molecules may also be used in the manufacture of a medicament for the treatment of diseases and conditions in which fibulin-like polypeptides are implicated such as those in which skin is damaged from aging, injuries or the sun, or for restoring skin damaged from the same, also multiple sclerosis, cancer, bone, joint or ligament reconstruction after fractures or lesions, osteoarthritis, rheumatoid arthritis, osteoporosis, cardiovascular diseases and fibrosis (including liver fibrosis and hepatitis).

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patient and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

A preferred method for detecting polypeptides of the first aspect of the invention comprises the steps of: (a) contacting a ligand, such as an antibody, of the sixth aspect of the invention with a biological sample under conditions suitable for the formation of a ligand-polypeptide complex; and (b) detecting said complex.

A number of different such methods according to the ninth aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

In a tenth aspect, the invention provides for the use of a polypeptide of the first aspect of the invention as a fibulin-like protein. Suitable uses include use as a secreted glycoprotein, in particular in the context of tissue repair and remodeling, as a result of the ability of the protein to bind to extracellular matrix structures such as connective tissue fibers, basement membranes and blood clots through interacting with other extracellular matrix proteins such as fibronectin, laminins, nidogen, perlecan, fibrillin and fibrinogen.

In an eleventh aspect, the invention provides a pharmaceutical composition comprising a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in the manufacture of a medicament for the diagnosis or treatment of a disease or condition in which fibulin-like polypeptides are implicated such as those in which skin is damaged from aging, injuries or the sun, or for restoring skin damaged from the same, also multiple sclerosis, cancer, bone, joint or ligament reconstruction after fractures or lesions, osteoarthritis, rheumatoid arthritis, osteoporosis, cardiovascular diseases and fibrosis (including liver fibrosis and hepatitis). In a thirteenth aspect, the invention provides a method of treating a disease in a patient comprising administering to the patient a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention.

For diseases in which the expression of a natural gene encoding a polypeptide of the first aspect of the invention, or in which the activity of a polypeptide of the first aspect of the invention, is lower in a diseased patient when compared to the level of expression or activity in a healthy patient, the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

In a fourteenth aspect, the invention provides transgenic or knockout non-human animals that have been transformed to express higher, lower or absent levels of a polypeptide of the first aspect of the invention. Such transgenic animals are very useful models for the study of disease and may also be used in screening regimes for the identification of compounds that are effective in the treatment or diagnosis of such a disease.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification. The practice of the present invention will employ, unless otherwise indicated, conventional techniques of molecular biology, microbiology, recombinant DNA technology and immunology, which are within the skill of the those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

The first aspect of the invention includes variants of the amino acid sequence recited in SEQ ID NO: 2 or SEQ ID NO: 4, wherein any amino acid specified in the chosen sequence is non-conservatively substituted, provided that no more than 15% of the amino acid residues in the sequence are so changed. Protein sequences having the indicated number of non-conservative substitutions can be identified using commonly available bioinformatic tools (Mulder N J and Apweiler R, 2002; Rehm B H, 2001).

In addition to such sequences, a series of polypeptides forms part of the disclosure of the invention. Being fibulin-like polypeptides known to go through maturation processes including the proteolytic removal of N-terminal sequences (by signal peptidases and other proteolytic enzymes), the present application also claims the mature form of the polypeptide whose sequence is recited in SEQ ID NO: 2. The sequence of this polypeptide is recited in SEQ ID NO: 4. Mature forms are intended to include any polypeptide showing fibulin-like activity and resulting from in vivo (by the expressing cells or animals) or in vitro (by modifying the purified polypeptides with specific enzymes) post-translational maturation processes. Other alternative mature forms can also result from the addition of chemical groups such as sugars or phosphates.

Other claimed polypeptides are the active variants of the amino acid sequences given by SEQ ID NO: 2 and SEQ ID NO: 4, wherein any amino acid specified in the chosen sequence is non-conservatively substituted, provided that no more than 15%, preferably no more that 10%, 5%, 3%, or 1%, of the amino acid residues in the sequence are so changed. The indicated percentage has to be measured over the novel amino acid sequences disclosed.

In accordance with the present invention, any substitution should be preferably a “conservative” or “safe” substitution, which is commonly defined a substitution introducing an amino acids having sufficiently similar chemical properties (eg a basic, positively charged amino acid should be replaced by another basic, positively charged amino acid), in order to preserve the structure and the biological function of the molecule.

The literature provide many models on which the selection of conservative amino acids substitutions can be performed on the basis of statistical and physico-chemical studies on the sequence and/or the structure of proteins (Rogov S I and Nekrasov A N, 2001). Protein design experiments have shown that the use of specific subsets of amino acids can produce foldable and active proteins, helping in the classification of amino acid “synonymous” substitutions which can be more easily accommodated in protein structure, and which can be used to detect functional and structural homologs and paralogs (Murphy L R et al., 2000). The groups of synonymous amino acids and the groups of more preferred synonymous amino acids are shown in Table I.

Active variants having comparable, or even improved, activity with respect of corresponding fibulin-like polypeptides may result from conventional mutagenesis technique of the encoding DNA, from combinatorial technologies at the level of encoding DNA sequence (such as DNA shuffling, phage display/selection), or from computer-aided design studies, followed by the validation for the desired activities as described in the prior art.

Specific, non-conservative mutations can be also introduced in the polypeptides of the invention with different purposes. Mutations reducing the affinity of the fibulin-like polypeptide may increase its ability to be reused and recycled, potentially increasing its therapeutic potency (Robinson C R, 2002). Immunogenic epitopes eventually present in the polypeptides of the invention can be exploited for developing vaccines (Stevanovic S, 2002), or eliminated by modifying their sequence following known methods for selecting mutations for increasing protein stability, and correcting them (van den Burg B and Eijsink V, 2002; WO 02/05146, WO 00/34317, WO 98/52976).

Further alternative polypeptides of the invention are active fragments, precursors, salts, or functionally-equivalent derivatives of the amino acid sequences described above.

Fragments should present deletions of terminal or internal amino acids not altering their function, and should involve generally a few amino acids, e.g., under ten, and preferably under three, without removing or displacing amino acids which are critical to the functional conformation of the proteins. Small fragments may form an antigenic determinant.

The “precursors” are compounds which can be converted into the compounds of present invention by metabolic and enzymatic processing prior or after the administration to the cells or to the body.

The term “salts” herein refers to both salts of carboxyl groups and to acid addition salts of amino groups of the polypeptides of the present invention. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid. Any of such salts should have substantially similar activity to the peptides and polypeptides of the invention or their analogs.

The term “derivatives” as herein used refers to derivatives which can be prepared from the functional groups present on the lateral chains of the amino acid moieties or on the amino- or carboxy-terminal groups according to known methods. Such molecules can result also from other modifications which do not normally alter primary sequence, for example in vivo or in vitro chemical derivativizatiod of polypeptides (acetylation or carboxylation), those made by modifying the pattern of phosphorylation (introduction of phosphotyrosine, phosphoserine, or phosphothreonine residues) or glycosylation (by exposing the polypeptide to mammalian glycosylating enzymes) of a peptide during its synthesis and processing or in further processing steps. Alternatively, derivatives may include esters or aliphatic amides of the carboxyl-groups and N-acyl derivatives of free amino groups or O-acyl derivatives of free hydroxyl-groups and are formed with acyl-groups as for example alcanoyl- or aryl-groups.

The generation of the derivatives may involve a site-directed modification of an appropriate residue, in an internal or terminal position. The residues used for attachment should they have a side-chain amenable for polymer attachment (i.e., the side chain of an amino acid bearing a functional group, e.g., lysine, aspartic acid, glutamic acid, cysteine, histidine, etc.). Alternatively, a residue having a side chain amenable for polymer attachment can replace an amino acid of the polypeptide, or can be added in an internal or terminal position of the polypeptide. Also, the side chains of the genetically encoded amino acids can be chemically modified for polymer attachment, or unnatural amino acids with appropriate side chain functional groups can be employed. The preferred method of attachment employs a combination of peptide synthesis and chemical ligation. Advantageously, the attachment of a water-soluble polymer will be through a biodegradable linker, especially at the amino-terminal region of a protein. Such modification acts to provide the protein in a precursor (or “pro-drug”) form, that, upon degradation of the linker releases the protein without polymer modification.

Polymer attachment may be not only to the side chain of the amino acid naturally occurring in a specific position of the antagonist or to the side chain of a natural or unnatural amino acid that replaces the amino acid naturally occurring in a specific position of the antagonist, but also to a carbohydrate or other moiety that is attached to the side chain of the amino acid at the target position. Rare or unnatural amino acids can be also introduced by expressing the protein in specifically engineered bacterial strains (Bock A, 2001).

All the above indicated variants can be natural, being identified in organisms other than humans, or artificial, being prepared by chemical synthesis, by site-directed mutagenesis techniques, or any other known technique suitable thereof, which provide a finite set of substantially corresponding mutated or shortened peptides or polypeptides which can be routinely obtained and tested by one of ordinary skill in the art using the teachings presented in the prior art.

The novel amino acid sequences disclosed in the present patent application can be used to provide different kind of reagents and molecules. Examples of these compounds are binding proteins or antibodies that can be identified using their full sequence or specific fragments, such as antigenic determinants. Peptide libraries can be used in known methods (Tribbick G, 2002) for screening and characterizing antibodies or other proteins binding the claimed amino acid sequences, and for identifying alternative forms of the polypeptides of the invention having similar binding properties.

The present patent application discloses also fusion proteins comprising any of the polypeptides described above. These polypeptides should contain protein sequence heterologous to the one disclosed in the present patent application, without significantly impairing the fibulin-like activity of the polypeptide and possibly providing additional properties. Examples of such properties are an easier purification procedure, a longer lasting half-life in body fluids, an additional binding moiety, the maturation by means of an endoproteolytic digestion, or extracellular localization. This latter feature is of particular importance for defining a specific group of fusion or chimeric proteins included in the above definition since it allows the claimed molecules to be localized in the space where not only isolation and purification of these polypeptides is facilitated, but also where generally fibulin-like polypeptides and their receptor interact.

Design of the moieties, ligands, and linkers, as well methods and strategies for the construction, purification, detection and use of fusion proteins are disclosed in the literature (Nilsson J et al., 1997; Methods Enzymol, Vol. 326-328, Academic Press, 2000). The preferred one or more protein sequences which can be comprised in the fusion proteins belong to these protein sequences: membrane-bound protein, immunoglobulin constant region, multimerization domains, extracellular proteins, signal peptide-containing proteins, export signal-containing proteins. Features of these sequences and their specific uses are disclosed in a detailed manner, for example, for albumin fusion proteins (WO 01/77137), fusion proteins including multimerization domain (WO 01/02440, WO 00/24782), immunoconjugates (Garnett M C, 2001), or fusion protein providing additional sequences which can be used for purifying the recombinant products by affinity chromatography (Constans A, 2002; Burgess R R and Thompson N E, 2002; Lowe C R et al., 2001; J. Bioch. Biophy. Meth., vol. 49 (1-3), 2001; Sheibani N, 1999).

The polypeptides of the invention can be used to generate and characterize ligands binding specifically to them. These molecules can be natural or artificial, very different from the chemical point of view (binding proteins, antibodies, molecularly imprinted polymers), and can be produced by applying the teachings in the art (WO 02/74938; Kuroiwa Y et al., 2002; Haupt K, 2002; van Dijk M A and van de Winkel J G, 2001; Gavilondo J V and Larrick J W, 2000). Such ligands can antagonize or inhibit the fibulin-like activity of the polypeptide against which they have been generated. In particular, common and efficient ligands are represented by extracellular domain of a membrane-bound protein or antibodies, which can be in the form monoclonal, polyclonal, humanized antibody, or an antigen binding fragment.

The polypeptides and the polypeptide-based derived reagents described above can be in alternative forms, according to the desired method of use and/or production, such as active conjugates or complexes with a molecule chosen amongst radioactive labels, fluorescent labels, biotin, or cytotoxic agents.

Specific molecules, such as peptide mimetics, can be also designed on the sequence and/or the structure of a polypeptide of the invention. Peptide mimetics (also called peptidomimetics) are peptides chemically modified at the level of amino acid side chains, of amino acid chirality, and/or of the peptide backbone. These alterations are intended to provide agonists or antagonists of the polypeptides of the invention with improved preparation, potency and/or pharmacokinetics features.

For example, when the peptide is susceptible to cleavage by peptidases following injection into the subject is a problem, replacement of a particularly sensitive peptide bond with a non-cleavable peptide mimetic can provide a peptide more stable and thus more useful as a therapeutic. Similarly, the replacement of an L-amino acid residue is a standard way of rendering the peptide less sensitive to proteolysis, and finally more similar to organic compounds other than peptides. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4-dinitrophenyl. Many other modifications providing increased potency, prolonged activity, easiness of purification, and/or increased half-life are disclosed in the prior art (WO 02/10195; Villain M et al., 2001).

Preferred alternative, synonymous groups for amino acids derivatives included in peptide mimetics are those defined in Table II. A non-exhaustive list of amino acid derivatives also include aminoisobutyric acid (Aib), hydroxyproline (Hyp), 1,2,3,4-tetrahydro-isoquinoline-3-COOH, indoline-2carboxylic acid, 4-difluoro-proline, L-thiazolidine-4-carboxylic acid, L-homoproline, 3,4-dehydro-proline, 3,4-dihydroxy-phenylalanine, cyclohexyl-glycine, and phenylglycine.

By “amino acid derivative” is intended an amino acid or amino acid-like chemical entity other than one of the 20 genetically encoded naturally occurring amino acids. In particular, the amino acid derivative may contain substituted or non-substituted, linear, branched, or cyclic alkyl moieties, and may include one or more heteroatoms. The amino acid derivatives can be made de novo or obtained from commercial sources (Calbiochem-Novabiochem AG, Switzerland; Bachem, USA).

Various methodologies for incorporating unnatural amino acids derivatives into proteins, using both in vitro and in vivo translation systems, to probe and/or improve protein structure and function are disclosed in the literature (Dougherty D A, 2000). Techniques for the synthesis and the development of peptide mimetics, as well as non-peptide mimetics, are also well known in the art (Golebiowski A et al., 2001; Hruby V J and Balse P M, 2000; Sawyer T K, in “Structure Based Drug Design”, edited by Veerapandian P, Marcel Dekker Inc., pg. 557-663, 1997).

Another object of the present invention are isolated nucleic acids encoding for the polypeptides of the invention having fibulin-like activity, the polypeptides binding to an antibody or a binding protein generated against them, the corresponding fusion proteins, or mutants having antagonistic activity as disclosed above. Preferably, these nucleic acids should comprise a DNA sequence selected from the group consisting of SEQ ID NO: 1, or the complement of said DNA sequences.

Alternatively, the nucleic acids of the invention should hybridize under high stringency conditions, or exhibit at least about 85% identity over a stretch of at least about 30 nucleotides, with a nucleic acid consisting of SEQ ID NO: 1, or be a complement of said DNA sequence.

The wording “high stringency conditions” refers to conditions in a hybridization reaction that facilitate the association of very similar molecules and consist in the overnight incubation at 60-65° C. in a solution comprising 50% formamide, 5×SSC (150 mM NaCl, 15 mM trisodium citrate), 50 mM sodium phosphate (pH 76), 5× Denhardt's solution, 10% dextran sulphate, and 20 microgram/ml denatured, sheared salmon sperm DNA, followed by washing the filters in 0.1×SSC at the same temperature.

These nucleic acids, including nucleotide sequences substantially the same, can be comprised in plasmids, vectors and any other DNA construct which can be used for maintaining, modifying, introducing, or expressing the encoding polypeptide. In particular, vectors wherein said nucleic acid molecule is operatively linked to expression control sequences can allow expression in prokaryotic or eukaryotic host cells of the encoded polypeptide.

The wording “nucleotide sequences substantially the same” includes all other nucleic acid sequences which, by virtue of the degeneracy of the genetic code, also code for the given amino acid sequences. In this sense, the literature provides indications on preferred or optimized codons for recombinant expression (Kane J F et al., 1995).

The nucleic acids and the vectors can be introduced into cells with different purposes, generating transgenic cells and organisms. A process for producing cells capable of expressing a polypeptide of the invention comprises genetically engineering cells with such vectors and nucleic acids.

In particular, host cells (e.g. bacterial cells) can be modified by transformation for allowing the transient or stable expression of the polypeptides encoded by the nucleic acids and the vectors of the invention. Alternatively, said molecules can be used to generate transgenic animal cells or non-human animals (by non-/homologous recombination or by any other method allowing their stable integration and maintenance), having enhanced or reduced expression levels of the polypeptides of the invention, when the level is compared with the normal expression levels. Such precise modifications can be obtained by making use of the nucleic acids of the inventions and of technologies associated, for example, to gene therapy (Meth. Enzymol., vol. 346, 2002) or to site-specific recombinases (Kolb A F, 2002). Model systems based on the fibulin-like polypeptides disclosed in the present patent application for the systematic study of their function can be also generated by gene targeting into human cell lines (Bunz F, 2002).

Gene silencing approaches may also be undertaken to down-regulate endogenous expression of a gene encoding a polypeptide of the invention. RNA interference (RNAi) (Elbashir, S M et al., Nature 2001, 411, 494-498) is one method of sequence specific post-transcriptional gene silencing that may be employed. Short dsRNA oligonucleotides are synthesised in vitro and introduced into a cell. The sequence specific binding of these dsRNA oligonucleotides triggers the degradation of target mRNA, reducing or ablating target protein expression.

Efficacy of the gene silencing approaches assessed above may be assessed through the measurement of polypeptide expression (for example, by Western blotting), and at the RNA level using TaqMan-based methodologies.

The polypeptides of the invention can be prepared by any method known in the art, including recombinant DNA-related technologies, and chemical synthesis technologies. In particular, a method for making a polypeptide of the invention may comprise culturing a host or transgenic cell as described above under conditions in which the nucleic acid or vector is expressed, and recovering the polypeptide encoded by said nucleic acid or vector from the culture. For example, when the vector expresses the polypeptide as a fusion protein with an extracellular or signal-peptide containing proteins, the recombinant product can be secreted in the extracellular space, and can be more easily collected and purified from cultured cells in view of further processing or, alternatively, the cells can be directly used or administered.

The DNA sequence coding for the proteins of the invention can be inserted and ligated into a suitable episomal or non-/homologously integrating vectors, which can be introduced in the appropriate host cells by any suitable means (transformation, transfection, conjugation, protoplast fusion, electroporation, calcium phosphate-precipitation, direct microinjection, etc.). Factors of importance in selecting a particular plasmid or viral vector include: the ease with which recipient cells that contain the vector, may be recognized and selected from those recipient cells which do not contain the vector; the number of copies of the vector which are desired in a particular host; and whether it is desirable to be able to “shuttle” the vector between host cells of different species.

The vectors should allow the expression of the isolated or fusion protein including the polypeptide of the invention in the Prokaryotic or Eukaryotic host cells under the control of transcriptional initiation/termination regulatory sequences, which are chosen to be constitutively active or inducible in said cell. A cell line substantially enriched in such cells can be then isolated to provide a stable cell line.

For Eukaryotic hosts (e.g. yeasts, insect, plant, or mammalian cells), different transcriptional and translational regulatory sequences may be employed, depending on the nature of the host. They may be derived form viral sources, such as adenovirus, bovine papilloma virus, Simian virus or the like, where the regulatory signals are associated with a particular gene which has a high level of expression. Examples are the TK promoter of the Herpes virus, the SV40 early promoter, the yeast gal4 gene promoter, etc. Transcriptional initiation regulatory signals may be selected which allow for repression and activation, so that expression of the genes can be modulated. The cells stably transformed by the introduced DNA can be selected by introducing one or more markers allowing the selection of host cells which contain the expression vector. The marker may also provide for phototrophy to an auxotropic host, biocide resistance, e.g. antibiotics, or heavy metals such as copper, or the like. The selectable marker gene can either be directly linked to the DNA gene sequences to be expressed, or introduced into the same cell by co-transfection.

Host cells may be either prokaryotic or eukaryotic. Preferred are eukaryotic hosts, e.g. mammalian cells, such as human, monkey, mouse, and Chinese Hamster Ovary (CHO) cells, because they provide post-translational modifications to proteins, including correct folding and glycosylation. Also yeast cells can carry out post-translational peptide modifications including glycosylation. A number of recombinant DNA strategies exist which utilize strong promoter sequences and high copy number of plasmids which can be utilized for production of the desired proteins in yeast. Yeast recognizes leader sequences in cloned mammalian gene products and secretes peptides bearing leader sequences (i.e., pre-peptides).

The above mentioned embodiments of the invention can be achieved by combining the disclosure provided by the present patent application on the sequence of novel fibulin-like polypeptides with the knowledge of common molecular biology techniques.

Many books and reviews provides teachings on how to clone and produce recombinant proteins using vectors and Prokaryotic or Eukaryotic host cells, such as some titles in the series “A Practical Approach” published by Oxford University Press (“DNA Cloning 2: Expression Systems”, 1995; “DNA Cloning 4: Mammalian Systems”, 1996; “Protein Expression”, 1999; “Protein Purification Techniques”, 2001).

Moreover, updated and more focused literature provides an overview of the technologies for expressing polypeptides in a high-throughput manner (Chambers S P, 2002; Coleman T A, et al., 1997), of the cell systems and the processes used industrially for the large-scale production of recombinant proteins having therapeutic applications (Andersen D C and Krummen L, 2002, Chu L and Robinson D K, 2001), and of alternative eukaryotic expression systems for expressing the polypeptide of interest, which may have considerable potential for the economic production of the desired protein, such the ones based on transgenic plants (Giddings G, 2001) or the yeast Pichia pastoris (Lin Cereghino G P et al., 2002). Recombinant protein products can be rapidly monitored with various analytical technologies during purification to verify the amount and the quantity of the expressed polypeptides (Baker K N et al., 2002), as well as to check if there is problem of bioequivalence and immunogenicity (Schellekens H, 2002; Gendel S M, 2002).

Totally synthetic fibulin-like polypeptides are disclosed in the literature and many examples of chemical synthesis technologies, which can be effectively applied for the fibulin-like polypeptides of the invention given their short length, are available in the literature, as solid phase or liquid phase synthesis technologies. for example, the amino acid corresponding to the carboxy-terminus of the peptide to be synthesized is bound to a support which is insoluble in organic solvents, and by alternate repetition of reactions, one wherein amino acids with their amino groups and side chain functional groups protected with appropriate protective groups are condensed one by one in order from the carboxy-terminus to the amino-terminus, and one where the amino acids bound to the resin or the protective group of the amino groups of the peptides are released, the peptide chain is thus extended in this manner. Solid phase synthesis methods are largely classified by the tBoc method and the Fmoc method, depending on the type of protective group used. Typically used protective groups include tBoc (t-butoxycarbonyl), Cl-Z (2-chlorobenzyloxycarbonyl), Br-Z (2-bromobenzyloxycarbonyl), Bzl (benzyl), Fmoc (9-fluorenylmethoxycarbonyl), Mbh (4,4′-dimethoxydibenzhydryl), Mtr (4-methoxy-2,3,6-trimethylbenzenesulphonyl), Trt (trityl), Tos (tosyl), Z (benzyloxycarbonyl) and C12-Bzl (2,6-dichlorobenzyl) for the amino groups; NO₂ (nitro) and Pmc (2,2,5,7,8-pentamethylchromane-6-sulphonyl) for the guanidino groups); and tBu (t-butyl) for the hydroxyl groups). After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method.

The purification of the polypeptides of the invention can be carried out by any one of the methods known for this purpose, i.e. any conventional procedure involving extraction, precipitation, chromatography, electrophoresis, or the like. A further purification procedure that may be used in preference for purifying the protein of the invention is affinity chromatography using monoclonal antibodies or affinity groups, which bind the target protein and which are produced and immobilized on a gel matrix contained within a column. Impure preparations containing the proteins are passed through the column. The protein will be bound to the column by heparin or by the specific antibody while the impurities will pass through. After washing, the protein is eluted from the gel by a change in pH or ionic strength. Alternatively, HPLC (High Performance Liquid Chromatography) can be used. The elution can be carried using a water-acetonitrile-based solvent commonly employed for protein purification.

The disclosure of the novel polypeptides of the invention, and the reagents disclosed in connection to them (antibodies, nucleic acids, cells) allows also to screen and characterize compounds that enhance or reduce their expression level into a cell or in an animal.

“Oligonucleotides” refers to either a single stranded polydeoxynucleotide or two complementary polydeoxynucleotide strands which may be chemically synthesized.

Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

The invention includes purified preparations of the compounds of the invention (polypeptides, nucleic acids, cells, etc.). Purified preparations, as used herein, refers to the preparations which contain at least 1%, preferably at least 5%, by dry weight of the compounds of the invention.

The present patent application discloses a series of novel fibulin-like polypeptides and of related reagents having several possible applications. In particular, whenever an increase in the fibulin-like activity of a polypeptide of the invention is desirable in the therapy or in the prevention of a disease, reagents such as the disclosed fibulin-like polypeptides, the corresponding fusion proteins and peptide mimetics, the encoding nucleic acids, the expressing cells, or the compounds enhancing their expression can be used.

Therefore, the present invention discloses pharmaceutical compositions for the treatment or prevention of diseases needing an increase in the fibulin-like activity of a polypeptide of the invention, which contain one of the disclosed fibulin-like polypeptides, the corresponding fusion proteins and peptide mimetics, the encoding nucleic acids, the expressing cells, or the compounds enhancing their expression, as active ingredient. The process for the preparation of these pharmaceutical compositions comprises combining the disclosed fibulin-like polypeptides, the corresponding fusion proteins and peptide mimetics, the encoding nucleic acids, the expressing cells, or the compounds enhancing their expression, together with a pharmaceutically acceptable carrier. Methods for the treatment or prevention of diseases needing an increase in the fibulin-like activity of a polypeptide of the invention, comprise the administration of a therapeutically effective amount of the disclosed fibulin-like polypeptides, the is corresponding fusion proteins and peptide mimetics, the encoding nucleic acids, the expressing cells, or the compounds enhancing their expression.

Amongst the reagents disclosed in the present patent application, the ligands, the antagonists or the compounds reducing the expression or the activity of polypeptides of the invention have several applications, and in particular they can be used in the therapy or in the diagnosis of a disease associated to the excessive fibulin-like activity of a polypeptide of the invention.

Therefore, the present invention discloses pharmaceutical compositions for the treatment or prevention of diseases associated to the excessive fibulin-like activity of a polypeptide of the invention, which contain one of the ligands, antagonists, or compounds reducing the expression or the activity of such polypeptides, as active ingredient. The process for the preparation of these pharmaceutical compositions comprises combining the ligand, the antagonist, or the compound, together with a pharmaceutically acceptable carrier. Methods for the treatment or prevention of diseases associated to the excessive fibulin-like activity of the polypeptide of the invention, comprise the administration of a therapeutically effective amount of the antagonist, the ligand or of the compound.

The pharmaceutical compositions of the invention may contain, in addition to fibulin-like polypeptide or to the related reagent, suitable pharmaceutically acceptable carriers, biologically compatible vehicles and additives which are suitable for administration to an animal (for example, physiological saline) and eventually comprising auxiliaries (like excipients, stabilizers, adjuvants, or diluents) which facilitate the processing of the active compound into preparations which can be used pharmaceutically.

The pharmaceutical compositions may be formulated in any acceptable way to meet the needs of the mode of administration. For example, of biomaterials, sugar-macromolecule conjugates, hydrogels, polyethylene glycol and other natural or synthetic polymers can be used for improving the active ingredients in terms of drug delivery efficacy. Technologies and models to validate a specific mode of administration are disclosed in literature (Davis B G and Robinson M A, 2002; Gupta P et al., 2002; Luo B and Prestwich G D, 2001; Cleland J L et al., 2001; Pillai O and Panchagnula R, 2001).

Polymers suitable for these purposes are biocompatible, namely, they are non-toxic to biological systems, and many such polymers are known. Such polymers may be hydrophobic or hydrophilic in nature, biodegradable, non-biodegradable, or a combination thereof. These polymers include natural polymers (such as collagen, gelatin, cellulose, hyaluronic acid), as well as synthetic polymers (such as polyesters, polyorthoesters, polyanhydrides). Examples of hydrophobic non-degradable polymers include polydimethyl siloxanes, polyurethanes, polytetrafluoroethylenes, polyethylenes, polyvinyl chlorides, and polymethyl methaerylates. Examples of hydrophilic non-degradable polymers include poly(2-hydroxyethyl methacrylate), polyvinyl alcohol, poly(N-vinyl pyrrolidone), polyalkylenes, polyacrylamide, and copolymers thereof. Preferred polymers comprise as a sequential repeat unit ethylene oxide, such as polyethylene glycol (PEG).

Any accepted mode of administration can be used and determined by those skilled in the art to establish the desired blood levels of the active ingredients. For example, administration may be by various parenteral routes such as subcutaneous, intravenous, intradermal, intramuscular, intraperitoneal, intranasal, transdermal, oral, or buccal routes. The pharmaceutical compositions of the present invention can also be administered in sustained or controlled release dosage forms, including depot injections, osmotic pumps, and the like, for the prolonged administration of the polypeptide at a predetermined rate, preferably in unit dosage forms suitable for single administration of precise dosages.

Parenteral administration can be by bolus injection or by gradual perfusion over time. Preparations for parenteral administration include sterile aqueous or non-aqueous solutions, suspensions, and emulsions, which may contain auxiliary agents or excipients known in the art, and can be prepared according to routine methods. In addition, suspension of the active compounds as appropriate oily injection suspensions may be administered. Suitable lipophilic solvents or vehicles include fatty oils, for example, sesame oil, or synthetic fatty acid esters, for example, sesame oil, or synthetic fatty acid esters, for example, ethyl oleate or triglycerides. Aqueous injection suspensions that may contain substances increasing the viscosity of the suspension include, for example, sodium carboxymethyl cellulose, sorbitol, and/or dextran. Optionally, the suspension may also contain stabilizers. Pharmaceutical compositions include suitable solutions for administration by injection, and contain from about 0.01 to 99.99 percent, preferably from about 20 to 75 percent of active compound together with the excipient.

The wording “therapeutically effective amount” refers to an amount of the active ingredients that is sufficient to affect the course and the severity of the disease, leading to the reduction or remission of such pathology. The effective amount will depend on the route of administration and the condition of the patient.

The wording “pharmaceutically acceptable” is meant to encompass any carrier, which does not interfere with the effectiveness of the biological activity of the active ingredient and that is not toxic to the host to which is administered. For example, for parenteral administration, the above active ingredients may be formulated in unit dosage form for injection in vehicles such as saline, dextrose solution, serum albumin and Ringer's solution. Carriers can be selected also from starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the various oils, including those of petroleum, animal, vegetable or synthetic origin (peanut oil, soybean oil, mineral oil, sesame oil).

It is understood that the dosage administered will be dependent upon the age, sex, health, and weight of the recipient, kind of concurrent treatment, if any, frequency of treatment, and the nature of the effect desired. The dosage will be tailored to the individual subject, as is understood and determinable by one of skill in the art. The total dose required for each treatment may be administered by multiple doses or in a single dose. The pharmaceutical composition of the present invention may be administered alone or in conjunction with other therapeutics directed to the condition, or directed to other symptoms of the condition. Usually a daily dosage of active ingredient is comprised between 0.01 to 100 milligrams per kilogram of body weight per day. Ordinarily 1 to 40 milligrams per kilogram per day given in divided doses or in sustained release form is effective to obtain the desired results. Second or subsequent administrations can be performed at a dosage, which is the same, less than, or greater than the initial or previous dose administered to the individual.

Apart from methods having a therapeutic or a production purpose, several other methods can make use of the fibulin-like polypeptides and of the related reagents disclosed in the present patent application.

In a first example, a method is provided for screening candidate compounds effective to treat a disease related to a fibulin-like polypeptide of the invention, said method comprising:
(a) contacting host cells expressing such polypeptide, transgenic non-human animals, or transgenic animal cells having enhanced or reduced expression levels of the polypeptide, with a candidate compound and
(b) determining the effect of the compound on the animal or on the cell.

In a second example there is provided a method for identifying a candidate compound as an antagonist/inhibitor or agonist/activator of a polypeptide of the invention, the method comprising:
(a) contacting the polypeptide, the compound, and a mammalian cell or a mammalian cell membrane; and
(b) measuring whether the molecule blocks or enhances the interaction of the polypeptide, or the response that results from such interaction, with the mammalian cell or the mammalian cell membrane.

In a third example, a method for determining the activity and/or the presence of the polypeptide of the invention in a sample, can detect either the polypeptide or the encoding RNA/DNA. Thus, such a method comprises:
(a) providing a protein-containing sample;
(b) contacting said sample with a ligand of the invention; and
(c) determining the presence of said ligand bound to said polypeptide, thereby determining the activity and/or the presence of polypeptide in said sample.

In an alternative, the method comprises:
(a) providing a nucleic acids-containing sample;
(b) contacting said sample with a nucleic acid of the invention; and
(c) determining the hybridization of said nucleic acid with a nucleic acid into the sample, thereby determining the presence of the nucleic acid in the sample.

In this sense, a primer sequence derived from the nucleotide sequence presented in SEQ ID NO:1 can be used as well for determining the presence or the amount of a transcript or of a nucleic acid encoding a polypeptide of invention in a sample by means of Polymerase Chain Reaction amplification.

A further object of the present invention are kits for measuring the activity and/or the presence of fibulin-like polypeptide of the invention in a sample comprising one or more of the reagents disclosed in the present patent application: a fibulin-like polypeptide of the invention, an antagonist, ligand or peptide mimetic, an isolated nucleic acid or the vector, a pharmaceutical composition, an expressing cell, or a compound increasing or decreasing the expression levels.

Such kits can be used for in vitro diagnostic or screenings methods, and their actual composition should be adapted to the specific format of the sample (e.g. biological sample tissue from a patient), and the molecular species to be measured. For example, if it is desired to measure the concentration of the fibulin-like polypeptide, the kit may contain an antibody and the corresponding protein in a purified form to compare the signal obtained in Western blot. Alternatively, if it is desired to measure the concentration of the transcript for the fibulin-like polypeptide, the kit may contain a specific nucleic acid probe designed on the corresponding ORF sequence, or may be in the form of nucleic acid array containing such probe. The kits can be also in the form of protein-, peptide mimetic-, or cell-based microarrays (Templin M F et al., 2002; Pellois J P et al., 2002; Blagoev B and Pandey A, 2001), allowing high-throughput proteomics studies, by making use of the proteins, peptide mimetics and cells disclosed in the present patent application.

The present patent application discloses novel fibulin-like polypeptides and a series of related reagents that may be useful, as active ingredients in pharmaceutical compositions appropriately formulated, in the treatment or prevention of diseases and conditions in which fibulin-like polypeptides are implicated such those in which skin is damaged from aging, injuries or the sun, or for restoring skin damaged from the same, also multiple sclerosis, cancer, bone, joint or ligament reconstruction after fractures or lesions, osteoarthritis, rheumatoid arthritis, osteoporosis, cardiovascular diseases and fibrosis (including liver fibrosis and hepatitis).

The therapeutic applications of the polypeptides of the invention and of the related reagents can be evaluated (in terms or safety, pharmacokinetics and efficacy) by the means of the in vivo/in vitro assays making use of animal cell, tissues and or by the means of in silico/computational approaches (Johnson D E and Wolfgang G H, 2000), known for the validation of fibulin-like polypeptides and other biological products during drug discovery and preclinical development.

The invention will now be described with reference to the specific embodiments by means of the following Examples, which should not be construed as in any way limiting the present invention. The content of the description comprises all modifications and substitutions which can be practiced by a person skilled in the art in light of the above teachings and, therefore, without extending beyond the meaning and purpose of the claims.

TABLE I

| Amino | | More Preferred |
|---|---|---|
| Acid | Synonymous Groups | Synonymous Groups |
| Ser | Gly, Ala, Ser, Thr, Pro | Thr, Ser |
| Arg | Asn, Lys, Gln, Arg, His | Arg, Lys, His |
| Leu | Phe, Ile, Val, Leu, Met | Ile, Val, Leu, Met |
| Pro | Gly, Ala, Ser, Thr, Pro | Pro |
| Thr | Gly, Ala, Ser, Thr, Pro | Thr, Ser |
| Ala | Gly, Thr, Pro, Ala, Ser | Gly, Ala |
| Val | Met, Phe, Ile, Leu, Val | Met, Ile, Val, Leu |
| Gly | Ala, Thr, Pro, Ser, Gly | Gly, Ala |
| Ile | Phe, Ile, Val, Leu, Met | Ile, Val, Leu, Met |
| Phe | Trp, Phe, Tyr | Tyr, Phe |
| Tyr | Trp, Phe, Tyr | Phe, Tyr |
| Cys | Ser, Thr, Cys | Cys |
| His | Asn, Lys, Gln, Arg, His | Arg, Lys, His |
| Gln | Glu, Asn, Asp, Gln | Asn, Gln |
| Asn | Glu, Asn, Asp, Gln | Asn, Gln |
| Lys | Asn, Lys, Gln, Arg, His | Arg, Lys, His |
| Asp | Glu, Asn, Asp, Gln | Asp, Glu |
| Glu | Glu, Asn, Asp, Gln | Asp, Glu |
| Met | Phe, Ile, Val, Leu, Met | Ile, Val, Leu, Met |
| Trp | Trp, Phe, Tyr | Trp |

TABLE II

| Amino | |
|---|---|
| Acid | Synonymous Groups |
| Ser | D-Ser, Thr, D-Thr, allo-Thr, Met, D-Met, Met(O), D-Met(O), L- |
| | Cys, D-Cys |
| Arg | D-Arg, Lys, D-Lys, homo-Arg, D-homo-Arg, Met, Ile, D-.Met, |
| | D-Ile, Orn, D-Orn |
| Leu | D-Leu, Val, D-Val, AdaA, AdaG, Leu, D-Leu, Met, D-Met |
| Pro | D-Pro, L-I-thioazolidine-4-carboxylic acid, D-or L-1- |
| | oxazolidine-4-carboxylic acid |
| Thr | D-Thr, Ser, D-Ser, allo-Thr, Met, D-Met, Met(O), D-Met(O), |
| | Val, D-Val |
| Ala | D-Ala, Gly, Aib, B-Ala, Acp, L-Cys, D-Cys |
| Val | D-Val, Leu, D-Leu, Ile, D-Ile, Met, D-Met, AdaA, AdaG |
| Gly | Ala, D-Ala, Pro, D-Pro, Aib, .beta.-Ala, Acp |
| Ile | D-Ile, Val, D-Val, AdaA, AdaG, Leu, D-Leu, Met, D-Met |
| Phe | D-Phe, Tyr, D-Thr, L-Dopa, His, D-His, Trp, D-Trp, |
| | Trans-3,4, or 5-phenylproline, AdaA, AdaG, cis-3,4, or |
| | 5-phenylproline, Bpa, D-Bpa |
| Tyr | D-Tyr, Phe, D-Phe, L-Dopa, His, D-His |
| Cys | D-Cys, S—Me-Cys, Met, D-Met, Thr, D-Thr |
| Gln | D-Gln, Asn, D-Asn, Glu, D-Glu, Asp, D-Asp |
| Asn | D-Asn, Asp, D-Asp, Glu, D-Glu, Gln, D-Gln |
| Lys | D-Lys, Arg, D-Arg, homo-Arg, D-homo-Arg, Met, D-Met, Ile, |
| | D-Ile, Orn, D-Orn |
| Asp | D-Asp, D-Asn, Asn, Glu, D-Glu, Gln, D-Gln |
| Glu | D-Glu, D-Asp, Asp, Asn, D-Asn, Gln, D-Gln |
| Met | D-Met, S—Me-Cys, Ile, D-Ile, Leu, D-Leu, Val, D-Val |

### EXAMPLES

### Example 1

Sequences of EGF protein domains from the ASTRAL database (Brenner S E et al. “The ASTRAL compendium for protein structure and sequence analysis” Nucleic Acids Res. 2000 Jan. 1; 28 (1): 254-6) were used to search for homologous protein sequences in genes predicted from human genome sequence (Celera database). The protein sequences were obtained from the gene predictions and translations thereof as generated by one of three programs: the Genescan (Burge C, Karlin S., “Prediction of complete gene structures in human genomic DNA, J Mol Biol. 1997 Apr. 25;268(1):78-94) Grail (Xu Y, Uberbacher E C., <<(Automated gene identification in large-scale genomic sequences”, J Comput Biol. 1997 Fall;4(3):325-38) and Fgenesh (Proprietary Celera software).

The sequence profiles of the EGF domains were generated using PIMAII (Profile Induced Multiple Alignment; Boston University software, version II, Das S and Smith T F 2000), an algorithm that aligns homologous sequences and generates a sequence profile. The homology was detected using PIMAII that generates global-local alignments between a query profile and a hit sequence. In this case the algorithm was used with the profile of the EGF functional domain as a query. PIMAII compares the query profile to the database of gene predictions translated into protein sequence and can therefore identify a match to a DNA sequence that contains that domain. Further comparison by BLAST (Basic Local Alignment Search Tool; NCBI version 2) of the sequence with known EFG containing proteins identified the closets homolog (Gish W, States D J. “Identification of protein coding regions by database similarity search.”, Nat Genet. 1993 March;3(3):266-72; Pearson W R, Miller W., “Dynamic programming algorithms for biological sequence comparison.”, Methods Enzymol. 1992;210:575-601; Altschul S F et al., “Basic local alignment search tool”, J Mol Biol. 1990 Oct. 5;215(3):403-10). PIMAII parameters used for the detection were the PIMA prior amino acids probability matrix and a Z-cutoff score of 10. BLAST parameters used were: Comparison matrix=BLOSUM62; word length=3; .E value cutoff=10; Gap opening and extension=default; No filter.

Once the functional domain was identified in the sequence, the genes were re-predicted with the genewise algorithm using the sequence of the closets homolog (Birney E et al., “PairWise and SearchWise: finding the optimal alignment in a simultaneous comparison of a protein profile against all DNA translation frames.”, Nucleic Acids Res. 1996 Jul. 15;24(14):2730-9)

The profiles for homologous EGF domains were generated automatically using the PSI-BLAST (Altshul et al 1997) scripts written in PERL (Practical Extraction and Report Language) and PIMAII.

A total of 55 predicted genes out of the 464 matching the original query generated on the basis of EGF domain profiles were selected since they were judged as potentially novel.

The novelty of the protein sequences was finally assessed by searching protein databases (SwissProt/Trembl, Human IPI and Derwent GENESEQ) using BLAST and a specific annotation has been attributed on the basis of amino acid sequence homology.

### Example 2

One sequence isolated by the methodology set out in Example 1 is that referred to herein as SCS0007. The homology exhibited by the SCS0007 sequence with Fibrillin 2 (J. Cell Biol. 124:855-863(1994) is very low (less than 40%) and limited to a calcium binding, EGF-like domain.

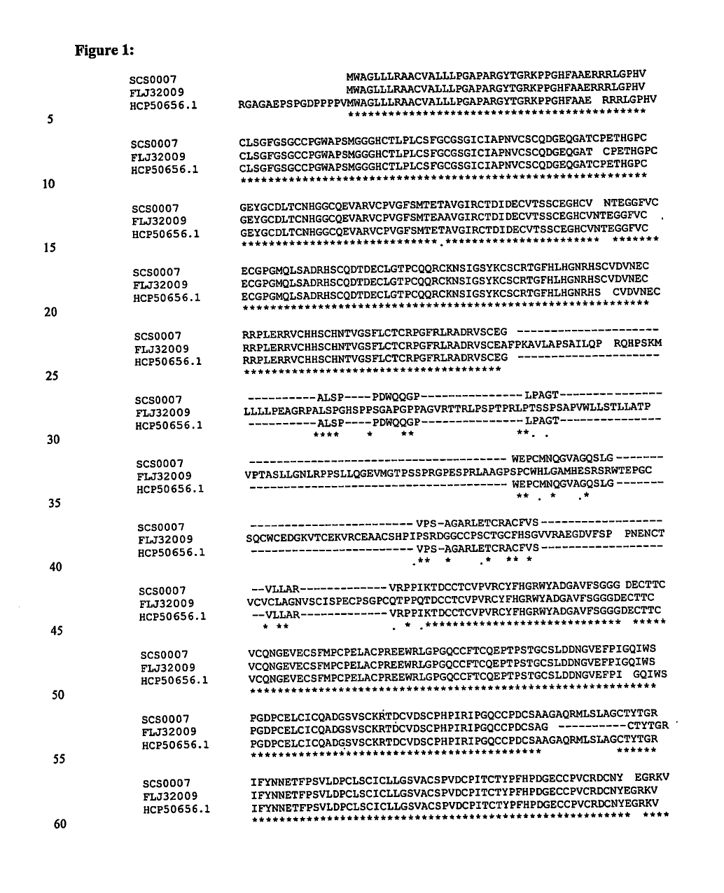

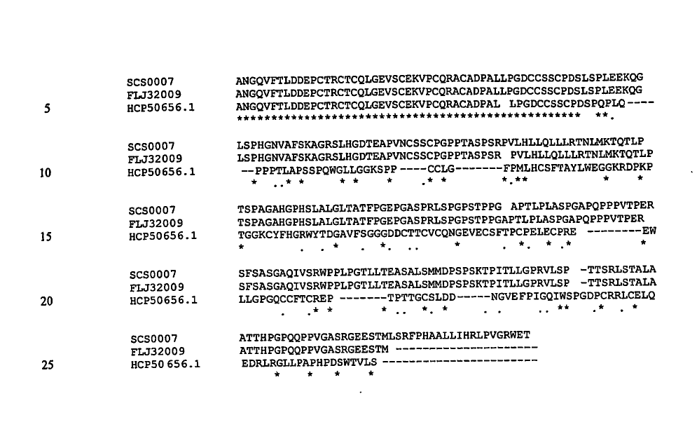

The most similar known polypeptide sequences are two predicted proteins, with accession numbers FLJ32009 (EMBL) and HCP50656.1 (CELERA), of which the submitted sequence appears as a hybrid. The protein can be roughly divided into four parts, where the first and the third are common to all the three sequences, the second one is identical in SCS0007 and CELERA, and the fourth one is identical in SCS0007 and EMBL (see FIG. 1).

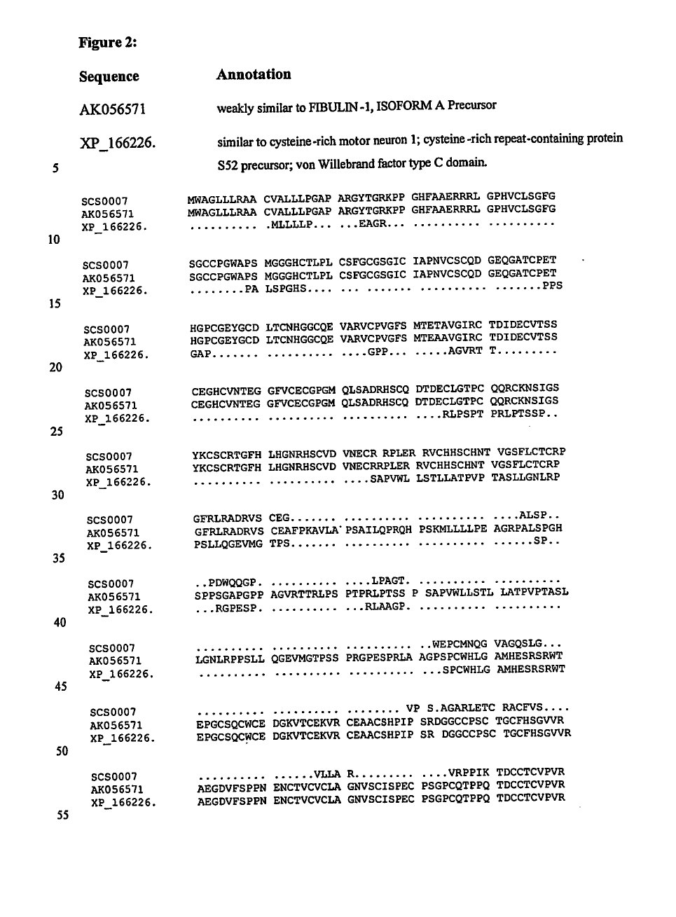

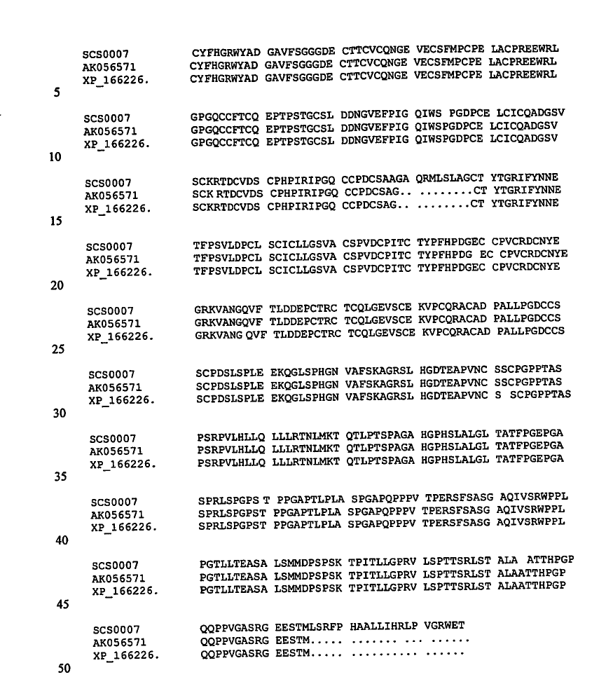

A further alignment is given in FIG. 2 along with the corresponding annotations. Both sequences were sourced from Genbank.

### REFERENCES

Andersen D C and Krummen L, Curr Opin Biotechnol, 13: 117-23, 2002.
Baker K N et al., Trends Biotechnol, 20: 149-56, 2002.
Blagoev B and Pandey A, Trends Biochem Sci, 26: 639-41, 2001.
Bock A, Science, 292: 453-4, 2001.
Bunz F, Curr Opin Oncol, 14: 73-8, 2002.
Burgess R R and Thompson N E, Curr Opin Biotechnol, 12: 450-4, 2001.
Chambers S P, Drug Disc Today, 14: 759-765, 2002.
Chu L and Robinson D K, Curr Opin Biotechnol, 13: 304-8, 2001.
Cleland J L et al., Curr Opin Biotechnol, 12: 212-9, 2001.
Coleman R A et al., Drug Discov Today, 6: 1116-1126, 2001.
Constans A, The Scientist, 16(4): 37, 2002.
Davis B G and Robinson M A, Curr Opin Drug Discov Devel, 5: 279-88, 2002.
Dougherty D A, Curr Opin Chem Biol, 4: 645-52, 2000.
Garnett M C, Adv Drug Deliv Rev, 53: 171-216, 2001.
Gavilondo J V and Larrick J W, Biotechniques, 29: 128-136, 2000.
Gendel S M, Ann N Y Acad Sci, 964: 87-98, 2002.
Giddings G, Curr Opin Biotechnol, 12: 450-4, 2001.
Golebiowski A et al., Curr Opin Drug Discov Devel, 4: 428-34, 2001.
Gupta P et al., Drug Discov Today, 7: 569-579, 2002.
Haupt K, Nat Biotechnol, 20: 884-885, 2002.
Hruby V J and Balse P M, Curr Med Chem, 7: 945-70, 2000.
Johnson D E and Wolfgang G H, Drug Discov Today, 5: 445-454, 2000.
Kane J F, Curr Opin Biotechnol, 6: 494-500, 1995.
Kolb A F, Cloning Stem Cells, 4: 65-80, 2002.
Kuroiwa Y et al., Nat Biotechnol, 20: 889-94, 2002.
Lin Cereghino G P et al., Curr Opin Biotechnol, 13: 329-332, 2001.
Lowe C R et al., J Biochem Biophys Methods, 49: 561-74, 2001.
Luo B and Prestwich G D, Exp Opin Ther Patents, 11: 1395-1410, 2001.
Mulder N J and Apweiler R, Genome Biol, 3(1):REVIEWS2001, 2002
Nilsson J et al., Protein Expr Purif, 11: 1-16, 1997.
Pearson W R and Miller W, Methods Enzymol, 210: 575-601, 1992.
Pellois J P et al., Nat Biotechnol, 20: 922-6, 2002.
Pillai O and Panchagnula R, Cur Opin Chem Biol, 5: 447-451, 2001
Rehm B H, Appl Microbiol Biotechnol, 57: 579-92, 2001.
Robinson C R, Nat Biotechnol, 20: 879-880, 2002.
Rogov S I and Nekrasov A N, Protein Eng, 14: 459-463, 2001.
Schellekens H, Nat Rev Drug Discov, 1: 457-62, 2002
Sheibani N, Prep Biochem Biotechnol, 29: 77-90, 1999.
Stevanovic S, Nat Rev Cancer, 2: 514-20, 2002.
Templin M F et al., Trends Biotechnol, 20: 160-6, 2002.
Tribbick G, J Immunol Methods, 267: 27-35, 2002.
van den Burg B and Eijsink V, Curr Opin Biotechnol, 13: 333-337, 2002.
van Dijk M A and van de Winkel J G, Curr Opin Chem Biol, 5: 368-74, 2001.
Villain M et al., Chem Biol, 8: 673-9, 2001.

1-41. (canceled)

42. A composition of matter comprising:
a) an isolated polypeptide having fibulin-like activity selected from the group consisting of: 1) the amino acid sequence recited in SEQ ID NO: 2; 2) the mature form of the polypeptide whose sequence is recited in SEQ ID NO: 2 (SEQ ID NO:4); 3) active variants of the amino acid sequence of SEQ ID NO: 2, wherein any amino acid specified in the chosen sequence is non-conservatively substituted, provided that no more than 15% of the amino acid residues in the sequence are so changed; and 4) the active fragment, precursor, salt, or derivative of the amino acid sequences given in 1), 2), or 3);
b) an isolated polypeptide that is a naturally occurring allelic variant of the sequence given by SEQ ID NO: 2 or SEQ ID NO:4;
c) an isolated polypeptide that is a naturally occurring allelic variant of the sequence given by SEQ ID NO: 2 or SEQ ID NO:4, wherein the variant is the translation of a single nucleotide polymorphism;
d) a polypeptide as set forth in a) or b) or c), wherein the polypeptide binds specifically an antibody or a binding protein generated against SEQ ID NO: 2 or SEQ ID NO:4 or a fragment thereof;
e) a fusion protein comprising a polypeptide as set forth in a) or b) or c) or d);
f) an antagonist of a polypeptide as set forth in a) or b) or c) or d), wherein said antagonist comprises an amino acid sequence resulting from the non-conservative substitution, the deletion or both the non-conservative substitution and deletion of one or more residues into the corresponding polypeptide;
g) a ligand which binds specifically to a polypeptide a) or b) or c) or d);
h) a polypeptide a) or b) or c) or d) or e), wherein said polypeptides are in the form of active conjugates or complexes with a molecule chosen from radioactive labels, fluorescent labels, biotin, or cytotoxic agents;
i) a peptide mimetic designed on the sequence or the structure or the sequence and structure of a polypeptide as set forth in a) or b) or c) or d);
j) an isolated nucleic acid encoding for an isolated polypeptide selected from the group consisting of:
1) polypeptides as set forth in a) or b) or c) or d);
2) a fusion protein comprising a polypeptide as set forth in a) or b) or c) or d); or
3) an antagonist of a polypeptide as set forth in a) or b) or c) or d), wherein said antagonist comprises an amino acid sequence resulting from the non-conservative substitution, the deletion or both the non-conservative substitution and deletion of one or more residues into the corresponding polypeptide;
k) an isolated nucleic acid sequence consisting of SEQ ID NO: 1, or a complement of said DNA sequence;
l) a purified nucleic acid which:
1) hybridizes under high stringency conditions; or
2) exhibits at least about 85% identity over a stretch of at least about 30 nucleotides with a nucleic acid selected from the group consisting of SEQ ID NO: 1, or a complement of said DNA sequence;
m) a vector comprising a nucleic acid as set forth in j) or k) or l);
n) a polypeptide encoded by the nucleic acid of j) or k) or l);
o) a host cell comprising a vector or nucleic acid as set forth in j) or k) or l) or m);
p) a transgenic animal cell comprising a vector or nucleic acid as set forth inj) or k) or l) or m) and having enhanced or reduced expression levels of a polypeptide as set forth in a) or b) or c) or d);
q) a transgenic non-human animal that has been transformed to have enhanced or reduced expression levels of a polypeptide as set forth in a) or b) or c) or d);
r) a compound that enhances the expression level of a polypeptide as set forth in a) or b) or c) or d) in a cell or animal; or
s) a compound that reduces the expression level of a polypeptide as set forth in a) or b) or c) or d) in a cell or animal.

43. The composition of matter according to claim 42, wherein said composition of matter comprises a polypeptide, peptide mimetic, nucleic acid, cell, or compound that enhances or reduces the expression of a polypeptide and a pharmaceutically acceptable carrier.

44. The composition of matter according to claim 42, wherein the fusion protein further comprises one or more amino acid sequence selected from the protein sequences: membrane-bound protein, immunoglobulin constant region, multimerization domains, extracellular proteins, signal peptide-containing proteins, or export signal-containing proteins.

45. The composition of matter according to claim 42, wherein the ligand antagonizes or inhibits the fibulin-like activity of a polypeptide.

46. The composition of matter according to claim 45, wherein the ligand is a monoclonal antibody, a polyclonal antibody, a humanized antibody, an antigen binding fragment, or the extracellular domain of a membrane-bound protein.

47. The composition of matter according to claim 42, wherein said vector comprises a nucleic acid molecule that is operatively linked to expression control sequences allowing expression in prokaryotic or eukaryotic host cells of the encoded polypeptide.

48. A method for determining the activity and/or the presence of a fibulin-like polypeptide in a sample comprising:
a) providing a protein-containing sample;
b) contacting said sample with a ligand that specifically binds to a polypeptide comprising:
1) an isolated polypeptide having fibulin-like activity selected from the group consisting of: i) the amino acid sequence recited in SEQ ID NO: 2; ii) the mature form of the polypeptide whose sequence is recited in SEQ ID NO: 2 (SEQ ID NO:4); iii) active variants of the amino acid sequence of SEQ ID NO: 2, wherein any amino acid specified in the chosen sequence is non-conservatively substituted, provided that no more than 15% of the amino acid residues in the sequence are so changed; and iv) the active fragment, precursor, salt, or derivative of the amino acid sequences given in i), ii), or iii);
2) an isolated polypeptide that is a naturally occurring allelic variant of the sequence given by SEQ ID NO: 2 or SEQ ID NO:4;
3) an isolated polypeptide that is a naturally occurring allelic variant of the sequence given by SEQ ID NO: 2 or SEQ ID NO:4, wherein the variant is the translation of a single nucleotide polymorphism; or
4) a polypeptide as set forth in a) or b) or c), wherein the polypeptide binds specifically an antibody or a binding protein generated against SEQ ID NO: 2 or SEQ ID NO:4 or a fragment thereof; and
c) determining the presence or said ligand bound to said polypeptide.

49. A method of using the composition of claim 42 for a) producing cells capable of expressing a polypeptide; b) making a polypeptide; c) the treatment of a disease; d) the preparation of pharmaceutical compositions; e) the screening candidate compounds; f) identifying a candidate compound; or g) determining the presence or the amount of a transcript or of a nucleic acid.

50. The method according to claim 49, wherein said method comprises genetically engineering cells with a vector or a nucleic acid comprising:
a) an isolated nucleic acid encoding for an isolated polypeptide selected from the group consisting of:
1) an isolated polypeptide having fibulin-like activity selected from the group consisting of: i) the amino acid sequence recited in SEQ ID NO: 2; ii) the mature form of the polypeptide whose sequence is recited in SEQ ID NO: 2 (SEQ ID NO:4); iii) active variants of the amino acid sequence of SEQ ID NO: 2, wherein any amino acid specified in the chosen sequence is non-conservatively substituted, provided that no more than 15% of the amino acid residues in the sequence are so changed; and iv) the active fragment, precursor, salt, or derivative of the amino acid sequences given in i), ii), or iii);
2) an isolated polypeptide that is a naturally occurring allelic variant of the sequence given by SEQ ID NO: 2 or SEQ ID NO:4;
3) an isolated polypeptide that is a naturally occurring allelic variant of the sequence given by SEQ ID NO: 2 or SEQ ID NO:4, wherein the variant is the translation of a single nucleotide polymorphism;
4) a polypeptide as set forth in 1) or 2) or 3), wherein the polypeptide binds specifically an antibody or a binding protein generated against SEQ ID NO: 2 or SEQ ID NO:4 or a fragment thereof; or
5) a fusion protein comprising a polypeptide as set forth in 1) or 2) or 3) or 4);
b) an isolated nucleic acid sequence consisting of SEQ ID NO: 1, or a complement of said DNA sequence;
c) a purified nucleic acid which:
1) hybridizes under high stringency conditions; or
2) exhibits at least about 85% identity over a stretch of at least about 30 nucleotides with a nucleic acid selected from the group consisting of SEQ ID NO: 1, or a complement of said DNA sequence; or
d) a vector comprising a nucleic acid as set forth in a) or b) or c).

51. The method according to claim 49, wherein said method comprises a method for making a polypeptide comprising culturing a transformed host cell under conditions in which the nucleic acid or vector is expressed, and recovering the polypeptide encoded by said nucleic acid or vector from the culture.

52. The method according to claim 49, wherein said method comprises the treatment of a disease needing an increase in the fibulin-like activity that comprises the administration of a therapeutically effective amount of a polypeptide, a peptide mimetic, a nucleic acid, a cell, or a compound as set forth in claim 42.

53. The method according to claim 49, wherein said method of treatment comprises the administration of a therapeutically effective amount of an antagonist, a ligand, or of a compound as set forth in claim 42.

54. The method according to claim 49, wherein said method for screening candidate compounds effective to treat a disease related to the fibulin-like polypeptides comprises:
a) contacting a cell, a transgenic animal cell, or a transgenic non-human animal having enhanced or reduced expression levels of the fibulin-like polypeptide, with a candidate compound and
b) determining the effect of the compound on the animal or on the cell.

55. The method according to claim 49, wherein said method for identifying a candidate compound as an antagonist/inhibitor or agonist/activator of a fibulin-like polypeptide comprises:
a) contacting said polypeptide, said compound, and a mammalian cell or a mammalian cell membrane capable of binding the polypeptide; and
b) measuring whether the molecule blocks or enhances the interaction of the polypeptide, or the response that results from such interaction, with the mammalian cell or the mammalian cell membrane.

56. The method according to claim 49, wherein said method for determining the presence or the amount of a transcript or of a nucleic acid encoding a fibulin-like polypeptide comprises:
a) providing a nucleic acids-containing sample;
b) contacting said sample with a nucleic acid as set forth in claim 42; and
c) determining the hybridization of said nucleic acid with a nucleic acid into the sample.


SEQUENCE LISTING (4 sequences)

SEQ ID NO: 1 - DNA, 2661 bp, homo sapiens
  misc_feature (50)..(2582): SCS0007 polynucleotide coding sequence
gcgcggcgcaggagccgagcccagcccgggggacccgccgccgccggtcatgtgggccggactgctccttcgggccgcctgtgtcgcgctcctgctgccgggggcaccagcccgaggctacaccgggaggaagccgcccgggcacttcgcggccgagagacgccgactgggcccccacgtctgcctctctgggtttgggagtggctgctgccctggctgggcgccctctatgggtggtgggcactgcaccctgcccctctgctccttcggctgtgggagtggcatctgcatcgctcccaatgtctgctcctgccaggatggagagcaaggggccacctgcccagaaacccatggaccatgtggggagtacggctgtgaccttacctgcaaccatggaggctgtcaggaggtggcccgagtgtgccccgtgggcttctcgatgacggagacagctgttggcatcaggtgtacagacattgacgaatgtgtaacctcctcctgcgagggccactgtgtgaacacagaaggtgggtttgtgtgcgagtgtgggccgggcatgcagctgtctgccgaccgccacagctgccaagacactgacgaatgcctagggactccctgtcagcagagatgtaaaaacagcattggcagctacaagtgttcctgtcgaactggcttccaccttcatggcaaccggcactcctgtgtagatgtaaacgagtgtcggaggccattggagaggcgagtctgtcaccattcctgccacaacaccgtgggcagcttcctatgcacatgccgacctggcttcaggctccgagctgaccgcgtgtcctgtgaaggggccctgagtccccccgactggcagcagggccctctccctgctggcacctgggagccatgcatgaatcaaggagtcgctggacagagcctgggtgttcccagtgctggtgcgaggctggaaacgtgtcgtgcatgtttcgtgagtgtccttttggcccgtgtgagaccccccataaagacggattgctgtacttgtgttccagtgagatgctatttccacggccggtggtacgcagacggggctgtgttcagtgggggtggtgacgagtgtaccacctgtgtttgccagaatggggaggtggagtgctccttcatgccctgccctgagctggcctgcccccgagaagagtggcggctgggccctgggcagtgttgcttcacctgccaggagcccacaccctcgacaggctgctctcttgacgacaacggggttgagtttccgattggacagatctggtcgcctggtgacccctgtgagttatgcatctgccaggcagatggctcggtgagctgcaagaggacagactgtgtggactcctgccctcacccgatccggatccctggacagtgctgcccagactgttcagcagctggtgctcagcgcatgctctctctggcaggctgcacctacacaggcagaatcttctataacaacgagaccttcccgtctgtgctggacccatgtctgagctgcatctgcctgctgggctcagtggcctgttcccccgtggactgccccatcacctgtacctaccctttccaccctgacggggagtgctgccccgtgtgccgagactgcaactacgagggaaggaaggtggcgaatggccaggtgttcaccttggatgatgaaccctgcacccggtgcacgtgccagctgggagaggtgagctgtgagaaggttccctgccagcgggcctgtgccgaccctgccctgcttcctggggactgctgctcttcctgtccagattccctgtctcctctggaagaaaagcaggggctctcccctcacggaaatgtggcattcagcaaagctggtcggagcctgcatggagacactgaggcccctgtcaactgtagctcctgtcctgggcccccgacagcatcaccctcgaggccggtgcttcatctcctccagctccttttaagaacgaacttgatgaaaacacagactttacctacaagcccggcaggagctcatggtccacactcactcgctttggggctgacagccactttcccaggggagcctggggcctcccctcgactctcaccagggccttcgacccctccaggagcccccactctacctctagcttccccaggggctcctcagccacctcctgtgactccagagcgctcgttctcagcctctggggcccagatagtgtccaggtggcctcctctgcctggcaccctcctgacggaagcttcagcactttccatgatggaccccagcccctcgaagacccccatcaccctcctcgggcctcgcgtgctttctcccaccacctctagactctccacagcccttgcagccaccacccaccctggcccccagcagcccccagtgggggcttctcggggggaagagtccaccatgttgtctcggtttccccatgctgcactgctcattcaccgcttacctgtgggaaggtgggaaacgtgaccccaagcccacaggtggtaagtgagcatccacctttaccccactgctggggagaaaagctggcaccaaattgtgactgggctggggaagggtctcctgtaagcacttggcggccttttatattgggagacttctttttatttttttccccaaga

SEQ ID NO: 2 - protein, 818 aa, homo sapiens
MWAGLLLRAACVALLLPGAPARGYTGRKPPGHFAAERRRLGPHVCLSGFGSGCCPGWAPSMGGGHCTLPLCSFGCGSGICIAPNVCSCQDGEQGATCPETHGPCGEYGCDLTCNHGGCQEVARVCPVGFSMTETAVGIRCTDIDECVTSSCEGHCVNTEGGFVCECGPGMQLSADRHSCQDTDECLGTPCQQRCKNSIGSYKCSCRTGFHLHGNRHSCVDVNECRRPLERRVCHHSCHNTVGSFLCTCRPGFRLRADRVSCEGALSPPDWQQGPLPAGTWEPCMNQGVAGQSLGVPSAGARLETCRACFVSVLLARVRPPIKTDCCTCVPVRCYFHGRWYADGAVFSGGGDECTTCVCQNGEVECSFMPCPELACPREEWRLGPGQCCFTCQEPTPSTGCSLDDNGVEFPIGQIWSPGDPCELCICQADGSVSCKRTDCVDSCPHPIRIPGQCCPDCSAAGAQRMLSLAGCTYTGRIFYNNETFPSVLDPCLSCICLLGSVACSPVDCPITCTYPFHPDGECCPVCRDCNYEGRKVANGQVFTLDDEPCTRCTCQLGEVSCEKVPCQRACADPALLPGDCCSSCPDSLSPLEEKQGLSPHGNVAFSKAGRSLHGDTEAPVNCSSCPGPPTASPSRPVLHLLQLLLRTNLMKTQTLPTSPAGAHGPHSLALGLTATFPGEPGASPRLSPGPSTPPGAPTLPLASPGAPQPPPVTPERSFSASGAQIVSRWPPLPGTLLTEASALSMMDPSPSKTPITLLGPRVLSPTTSRLSTALAATTHPGPQQPPVGASRGEESTMLSRFPHAALLIHRLPVGRWET

SEQ ID NO: 3 - DNA, 2397 bp, homo sapiens
gcccgaggctacaccgggaggaagccgcccgggcacttcgcggccgagagacgccgactgggcccccacgtctgcctctctgggtttgggagtggctgctgccctggctgggcgccctctatgggtggtgggcactgcaccctgcccctctgctccttcggctgtgggagtggcatctgcatcgctcccaatgtctgctcctgccaggatggagagcaaggggccacctgcccagaaacccatggaccatgtggggagtacggctgtgaccttacctgcaaccatggaggctgtcaggaggtggcccgagtgtgccccgtgggcttctcgatgacggagacagctgttggcatcaggtgtacagacattgacgaatgtgtaacctcctcctgcgagggccactgtgtgaacacagaaggtgggtttgtgtgcgagtgtgggccgggcatgcagctgtctgccgaccgccacagctgccaagacactgacgaatgcctagggactccctgtcagcagagatgtaaaaacagcattggcagctacaagtgttcctgtcgaactggcttccaccttcatggcaaccggcactcctgtgtagatgtaaacgagtgtcggaggccattggagaggcgagtctgtcaccattcctgccacaacaccgtgggcagcttcctatgcacatgccgacctggcttcaggctccgagctgaccgcgtgtcctgtgaaggggccctgagtccccccgactggcagcagggccctctccctgctggcacctgggagccatgcatgaatcaaggagtcgctggacagagcctgggtgttcccagtgctggtgcgaggctggaaacgtgtcgtgcatgtttcgtgagtgtccttttggcccgtgtgagaccccccataaagacggattgctgtacttgtgttccagtgagatgctatttccacggccggtggtacgcagacggggctgtgttcagtgggggtggtgacgagtgtaccacctgtgtttgccagaatggggaggtggagtgctccttcatgccctgccctgagctggcctgcccccgagaagagtggcggctgggccctgggcagtgttgcttcacctgccaggagcccacaccctcgacaggctgctctcttgacgacaacggggttgagtttccgattggacagatctggtcgcctggtgacccctgtgagttatgcatctgccaggcagatggctcggtgagctgcaagaggacagactgtgtggactcctgccctcacccgatccggatccctggacagtgctgcccagactgttcagcagctggtgctcagcgcatgctctctctggcaggctgcacctacacaggcagaatcttctataacaacgagaccttcccgtctgtgctggacccatgtctgagctgcatctgcctgctgggctcagtggcctgttcccccgtggactgccccatcacctgtacctaccctttccaccctgacggggagtgctgccccgtgtgccgagactgcaactacgagggaaggaaggtggcgaatggccaggtgttcaccttggatgatgaaccctgcacccggtgcacgtgccagctgggagaggtgagctgtgagaaggttccctgccagcgggcctgtgccgaccctgccctgcttcctggggactgctgctcttcctgtccagattccctgtctcctctggaagaaaagcaggggctctcccctcacggaaatgtggcattcagcaaagctggtcggagcctgcatggagacactgaggcccctgtcaactgtagctcctgtcctgggcccccgacagcatcaccctcgaggccggtgcttcatctcctccagctccttttaagaacgaacttgatgaaaacacagactttacctacaagcccggcaggagctcatggtccacactcactcgctttggggctgacagccactttcccaggggagcctggggcctcccctcgactctcaccagggccttcgacccctccaggagcccccactctacctctagcttccccaggggctcctcagccacctcctgtgactccagagcgctcgttctcagcctctggggcccagatagtgtccaggtggcctcctctgcctggcaccctcctgacggaagcttcagcactttccatgatggaccccagcccctcgaagacccccatcaccctcctcgggcctcgcgtgctttctcccaccacctctagactctccacagcccttgcagccaccacccaccctggcccccagcagcccccagtgggggcttctcggggggaagagtccaccatgttgtctcggtttccccatgctgcactgctcattcaccgcttacctgtgggaaggtgggaaacgtga

SEQ ID NO: 4 - protein, 798 aa, homo sapiens
ARGYTGRKPPGHFAAERRRLGPHVCLSGFGSGCCPGWAPSMGGGHCTLPLCSFGCGSGICIAPNVCSCQDGEQGATCPETHGPCGEYGCDLTCNHGGCQEVARVCPVGFSMTETAVGIRCTDIDECVTSSCEGHCVNTEGGFVCECGPGMQLSADRHSCQDTDECLGTPCQQRCKNSIGSYKCSCRTGFHLHGNRHSCVDVNECRRPLERRVCHHSCHNTVGSFLCTCRPGFRLRADRVSCEGALSPPDWQQGPLPAGTWEPCMNQGVAGQSLGVPSAGARLETCRACFVSVLLARVRPPIKTDCCTCVPVRCYFHGRWYADGAVFSGGGDECTTCVCQNGEVECSFMPCPELACPREEWRLGPGQCCFTCQEPTPSTGCSLDDNGVEFPIGQIWSPGDPCELCICQADGSVSCKRTDCVDSCPHPIRIPGQCCPDCSAAGAQRMLSLAGCTYTGRIFYNNETFPSVLDPCLSCICLLGSVACSPVDCPITCTYPFHPDGECCPVCRDCNYEGRKVANGQVFTLDDEPCTRCTCQLGEVSCEKVPCQRACADPALLPGDCCSSCPDSLSPLEEKQGLSPHGNVAFSKAGRSLHGDTEAPVNCSSCPGPPTASPSRPVLHLLQLLLRTNLMKTQTLPTSPAGAHGPHSLALGLTATFPGEPGASPRLSPGPSTPPGAPTLPLASPGAPQPPPVTPERSFSASGAQIVSRWPPLPGTLLTEASALSMMDPSPSKTPITLLGPRVLSPTTSRLSTALAATTHPGPQQPPVGASRGEESTMLSRFPHAALLIHRLPVGRWET